(19) United States
(12) Patent Application Publication
(10) Pub. No.: US 2014/0093502 A1
(43) Pub. Date: Apr. 3, 2014

(54) Immunosuppressive Combination and Its Use in the Treatment or Prophylaxis of Insulin-producing Cell Graft Rejection

(71) Applicants: Philip Lake, Parippany, NJ (US); Claudy Mullon, Acton, MA (US)

(72) Inventors: Philip Lake, Parippany, NJ (US); Claudy Mullon, Acton, MA (US)

(73) Assignee: Novartis AG, Basel (CH)

(21) Appl. No.: 14/098,179

(22) Filed: Dec. 5, 2013

Related U.S. Application Data
(63) Continuation of application No. 13/154,106, filed on Jun. 6, 2011.
Application No. 13/154,106 is a continuation of application No. 12/107,726, filed on Apr. 22, 2008, now abandoned.
Application No. 12/107,726 is a continuation of application No. 11/372,383, filed on Mar. 9, 2006, now abandoned.
Application No. 11/372,383 is a continuation of application No. 10/164,755, filed on Jun. 7, 2002, now abandoned.
(60) Provisional application No. 60/297,069, filed on Jun. 8, 2001.

(51) Int. Cl. A61K 39/395; A61K 31/439; A61K 38/17; A61K 31/137

(52) U.S. Cl. CPC A61K 39/3955; A61K 31/137; A61K 31/439; A61K 38/177
USPC 424/133.1

(57) ABSTRACT

A pharmaceutical combination comprising an accelerated lymphocyte homing agent in free form or in pharmaceutically acceptable salt form, and one or more compounds selected from the group consisting of an antibody to the IL-2 receptor, an immunosuppressive macrocyclic lactone and a soluble human complement inhibitor is used to treat or prevent insulin-producing cell graft rejection.

### FIELD OF THE INVENTION

The present invention relates to a method of treatment or prophylaxis of insulin-producing cell graft rejection, particularly pancreatic islet graft rejection.

### BACKGROUND OF THE INVENTION

Type 1 diabetes is caused by a progressive, autoimmune destruction of the insulin-producing β-cells within the islets of the pancreas. At present, multiple daily insulin injections, or insulin pump therapy, remain the treatments of choice for the majority of diabetic patients. Intensive insulin therapy can decrease the incidence of secondary complications, but the effect is not absolute and patients are at increased risk for serious episodes of hypoglycemia.

Islet transplantation is a significantly safer method for replacing the diseased glandular tissue in diabetics than pancreatic organ transplantation, and has been investigated for more than 10 years as a treatment for type 1 diabetes mellitus in patients with inadequate glucose control despite intensive insulin therapy.

The majority of islet transplant procedures have been performed in kidney graft recipients already receiving an immunosuppressive regimen consisting of antibody induction with antilymphocyte globulin and life-long treatment with ciclosporine, azathioprine and glucocorticoids, see Brendel et al., International Islet Transplant Registry Report, Giessen, Germany, pp. 1-20 (1999).

However, islet engraftment has been difficult to achieve with such an immunosuppressive regimen due to rejection, recurrent autoimmunity, primary non-function (PNF), and the diabetogenicity of conventional immunosuppressive drugs. In particular, proinflammatory mediators, produced by activated intrahepatic macrophages and endothelial cells subsequent to islet infusion, are detrimental to islet function and may lead to early islet loss or PNF of the graft.

Thus it has been estimated that over 10,000 islet equivalents (IEQ) per kg of recipient body weight are required in order to reproducibly achieve insulin independence in non-human primates (baboons, rhesus and cynomolgus monkeys), see Kenyon at al., Diabetes, Vol. 48, pp. 8132-8137 (1999); and humans, see Shapiro et al., New Engl. J. Med., Vol. 343, No. 4, pp. 230-238 (2000).

Yamasaki and co-workers have reported achieving prolonged islet allograft survival of up to 20 days in male rats rendered hyperglycemic with streptozotocin by pre-administration of FTY720 the day before and the day of grafting, Cell Transplantation, Vol. 7, No. 4, pp. 403-406 (1998).

Kenyon and co-workers demonstrated that islet transplantation can result in the reversal of hyperglycemia and in long-term insulin independence in humans and in several animal models of diabetes, including rodents, dogs, cynomolgus monkeys, rhesus monkeys and baboons (see Kenyon et al., supra), using an immunosuppressive regimen consisting of anti-CD154.

Shapiro and co-workers recently reported achieving favorable results in patients with type 1 diabetes and a history of severe hypoglycemia and metabolic instability who underwent islet transplantation in conjunction with a glucocorticoid-free immunosuppressive regimen consisting of rapamycin (i.e., sirolimus), tacrolimus and daclizumab (i.e., a humanized antibody to the IL-2 receptor). Seven out of seven patients who received islet allografts became insulin-independent. The longest reported patient follow up was 17 months post-transplantation, see Shapiro et al., supra.

However, there is still a need for an improved therapy to achieve improved insulin-producing cell engraftment, e.g., pancreatic islet engraftment with an improved quality of life.

### SUMMARY OF THE INVENTION

It has now been found that co-administration of an accelerated lymphocyte homing (“ALH”) agent with one or more immunosuppressive agents acting via a different mechanism than the ALH agent, to an islet graft recipient, provides an effective treatment or prophylaxis of pancreatic islet cell transplant rejection, and in particular enables type I diabetic transplant patients to achieve extended insulin independence.

### DETAILED DESCRIPTION OF THE INVENTION

In a particular embodiment the present invention comprises a method for the treatment or prophylaxis of insulin-producing cell graft rejection in an insulin-producing cell graft recipient comprising co-administering to the recipient an effective amount of an ALH agent and one or more compounds selected from the group consisting of an antibody to the IL-2 receptor, an immunosuppressive macrocyclic lactone, and a soluble human complement inhibitor. Preferably the co-administration therapy of the invention is glucocorticoid-free.

Preferably, the invention comprises combined administration of an ALH agent, an antibody to the IL-2 receptor and an immunosuppressive macrocyclic lactone. Optionally, such a treatment may additionally include administration of a soluble human complement inhibitor.

The combination therapy of the invention facilitates engraftment, sustained insulin independence, and long-term survival of insulin-producing cell allo- or xenografts. A particular advantage of the present therapy is in facilitating single-donor transplants, which are less clinically challenging than multiple-donor grafts, by effectively reducing the numbers of transplanted cells needed to provide functional insulin-producing cell mass in the patient. For example, the present therapy can reduce the required number of IEQ to 5,000 mg/kg per recipient, or less.

By “Insulin independence” is meant endogenous insulin production as determined after intravenous (i.v.) glucose tolerance test to the extent that the subject has normal glucose tolerance.

By “insulin-producing cell” are meant islets of Langerhans (of allo or xeno origin) and other cells such as suitable insulin-secreting cells or cell lines, e.g., stem cell derived or cloned insulin-secreting cells.

As alternative to the above, the present invention also provides:

1. Use of an ALH agent in free form or in pharmaceutically acceptable salt form in combination with one or more compounds selected from the group consisting of an antibody to the IL-2 receptor, an immunosuppressive macrocyclic lactone and a soluble human complement inhibitor, to treat or prevent insulin-producing cell graft rejection.

2. A pharmaceutical combination comprising: a) an ALH agent in free form or in pharmaceutically acceptable salt form; and b) one or more compounds elected from the group consisting of an antibody to the IL-2 receptor, an immunosuppressive macrocyclic lactone and a soluble human complement inhibitor.

The term “pharmaceutical combination” as used herein preferably includes a non-fixed combination, e.g., the active components are administered to a patient as separate entities either simultaneously, concurrently or sequentially with no specific limits, wherein such administration provides therapeutically effective levels of the components in the body of the patient. Each active component may be administered in the form of a pharmaceutical composition, e.g., the active component is associated with one or more pharmaceutically acceptable diluents or carriers therefor.

3. Use of a pharmaceutical combination as described above in a method as disclosed above.

4. Use of an ALH agent in free form or in pharmaceutically acceptable salt form, in the manufacture of a medicament for use in treating or preventing insulin-producing cell graft rejection in an insulin-producing cell graft recipient, in combination with one or more compounds selected from the group consisting of an antibody to the IL-2 receptor, an immunosuppressive macrocyclic lactone and a soluble human complement inhibitor.

Preferably the ALH agent is administered in combination with an immunosuppressive macrocyclic lactone, optionally together with a soluble human complement inhibitor; alternatively, the ALH agent may be administered in combination with an immunosuppressive macrocyclic lactone and an antibody to the IL-2 receptor, optionally together with a soluble human complement inhibitor.

The ALH agents of the invention are compounds which may be phosphorylated by sphingosine kinase and are in the phosphorylated form potent agonists at S1P receptors, thereby modulating lymphocyte trafficking, e.g., synthetic analogs of myriocin or ISP-1, a natural metabolite or the ascomycete Isaria sinclairli. Examples of an ALH agent include, e.g., 2-aminopropane 1-3-diol compounds, e.g., a compound of formula I

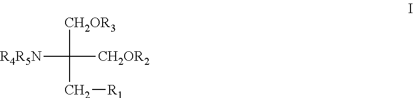

wherein
R₁ is an optionally substituted straight or branched carbon chain having 12-22 carbon atoms which may be optionally interrupted by an optionally substituted phenylene;
R₂ is H or a residue of formula

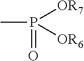

wherein
R₆ is H or C₁₋₄alkyl optionally substituted by 1, 2 or 3 halogen atoms, and R₇ is H or C₁₋₄alkyl optionally substituted by halogen;
R₃ is H or C₁₋₄alkyl; and
each of R₄ and R₅, independently, is H, C₁₋₄alkyl optionally substituted by halogen or acyl;

in free form or in pharmaceutically acceptable salt form.

When the carbon chain as R₁ is substituted, it is preferably substituted by halogen, nitro, amino, hydroxy or carboxy. When the carbon chain is interrupted by an optionally substituted phenylene, the carbon chain is preferably unsubstituted. When the phenylene moiety is substituted, it is preferably substituted by halogen, nitro, amino, methoxy, hydroxy or carboxy. Acyl may be a residue R—CO—, wherein R is C₁₋₅alkyl, C₃₋₅cycloalkyl, phenyl or phenyl-C₁₋₄alkyl.

Preferred compounds of formula I are those wherein R₁ is a straight or branched, preferably straight, chain alkyl having 13-20 carbon atoms, optionally substituted by nitro, halogen, amino, hydroxy or carboxy, and, more preferably those wherein R₁ is phenylalkyl substituted by a straight or branched C₆₋₁₄alkyl chain optionally substituted by halogen and the alkyl moiety is a C₁₋₆alkyl optionally substituted by hydroxy. More preferably, R₁ is phenyl-C₁₋₆alkyl substituted on the phenyl by a straight or branched, preferably straight, C₆₋₁₄alkyl chain. The C₆₋₁₄alkyl chain may be in ortho, meta or para, preferably in para.

Preferably each of R₂ to R₅ is H.

When the compounds of formula I have one or more asymmetric centers in the molecule, the present invention is to be understood as embracing the various optical isomers, as well as racemates, diastereoisomers and mixtures thereof are embraced.

Examples of pharmaceutically acceptable salts of the compounds of the formula I include salts with inorganic acids, such as hydrochloride, hydrobromide and sulfate; salts with organic acids, such as acetate, fumarate, maleate, benzoate, citrate, malate, methanesulfonate and benzenesulfonate salts; or, when appropriate, salts with metals, such as sodium, potassium, calcium and aluminium; salts with amines, such as triethylamine; and salts with dibasic amino acids, such as lysine. The compounds and salts of the methods of the present invention encompass hydrate and solvate forms.

A preferred compound of formula I is 2-amino-2-tetradecyl-1,3-propanediol. A particularly preferred ALH compound for use in the invention is FTY720, i.e., 2-amino-2-[2-(4-octylphenyl)ethyl]propane-1,3-diol in free form or in a pharmaceutically salt form, e.g., the hydrochloride, as shown:

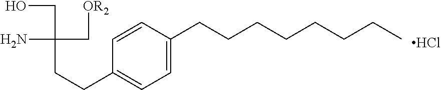

wherein
R₂ is H;
or its corresponding phosphate, wherein R₂ is

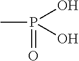

in free form or in a pharmaceutically acceptable salt form.

A disclosure of compounds, substituent groups, and variations included in the ALH-compounds of this invention and methods of preparing said compounds can be found in U.S. Pat. Nos. 5,604,229 and 6,004,565, in EP-A-1,002,792 and in WO 02/18395A, incorporated herein by reference in their entirety.

FTY720, a novel immunomodulator, increases the responsiveness of lymphocytes to homing chemokines. Náive cells are sequestered; CD4 and CD8 T-cells and B-cells from the blood are stimulated to migrate into lymph nodes (LN) and Peyer's patches (PP), and infiltration of cells into transplanted organs is inhibited. However, FTY720 does not impair lymphocyte activation, expansion and memory within the lymphoid system, and therefore does not suppress immunity to systemic infection.

The anti-IL-2 receptor antibody of the invention is preferably an antibody to the high affinity receptor for IL-2, i.e., CD25. Suitable antibodies comprise native or recombinant antibodies, and include recombinant chimeric or humanized antibodies, as well as recombinant single-chain polypeptides consisting of a native antibody binding (i.e., Fv) domain, e.g., basiliximab (Simulect™), which is a chimeric antibody comprising the variable region of murine monoclonal antibody CHI-621 and a human IgG1 region, see EP 449,769, incorporated herein by reference, or daclizumab (Zenapax®), see WO 90/07,861 incorporated herein by reference in its entirety. A particularly preferred antibody is basiliximab.

By “immunosuppressive macrocyclic lactone” is meant rapamycin, i.e., sirolimus, and immunosuppressant derivatives thereof. Of particular interest are rapamycin derivatives which are substituted in position 40 (or 42 or 43 depending on the nomenclature used), e.g., 40-O-substituted rapamycin derivatives as described in U.S. Pat. No. 5,258,369 and WO 94/09010 (incorporated herein by reference in their entirety), especially 40-O-alkylated rapamycin derivatives, e.g., wherein the 40-O-substituent is hydroxyalkylated, e.g., 40-O-(2-hydroxyethyl) rapamycin, i.e., everolimus, or derivatives substituted in position 40 and/or in other positions of the molecule, e.g., in position 28 and/or 16, including epimers thereof, and optionally further hydrogenated, e.g., as disclosed in WO 95/14023 and WO 99/15530 (incorporated herein by reference in their entirety), e.g., ABT578, or rapalogs as disclosed, e.g., in WO 98/02441 and WO 01/14387 (incorporated herein by reference in their entirety), e.g., AP23573. 40-O-(2-hydroxyethyl) rapamycin is particularly preferred.

Suitable soluble complement inhibitor includes, e.g., a C3/C5 inhibitor, e.g., a soluble complement receptor type I (CR1), TP-10, which is a recombinant protein that is a potent systemic inhibitor of the complement system, since it blocks both C3 and C5 activation by all three activation pathways (classical, alternative and lectin); and it is subsequent to C3 activation that the majority of complement-dependent effector mechanisms are recruited. Specifically, TP-10 binds C3b and C4b, activation fragments of the complement system, blocking their interaction with other proteins in the complement cascade and subsequently the formation of multi-molecular enzyme complexes which generate the biologically active protein fragments of complement. TP-10 also acts as a co-factor in the enzymatic degradation of C3b and C4b to their inactive forms.

TP-10 is a modified CR1 molecule lacking the transmembrane and cytoplasmic domains, e.g. as disclosed in WO 89/09220, incorporated herein by reference in its entirety. TP-10 is expressed by Chinese hamster ovary (CHO) cells in serum-free media and purified on anti-CR1 affinity columns and by HPLC. Administration of TP10 to islet transplant recipients was reported by Bennett, et. al., Diabetes (2000).

Other embodiments of a soluble complement receptor inhibitor suitable for use in the invention comprise TP-20, a combined complement and selectin inhibitor that integrates sCR1 (soluble complement receptor-1) with the sLex (sialyl Lewis x) carbohydrate in a single molecule; and TP-18, an sCR1 derivative inserted into a selectin-(receptor)-blocking carbohydrate.

Daily dosages of the therapeutic agents required in practicing the method of the present invention will vary, depending upon, for example, the ALH agent employed, the host, the mode of administration, the severity of the condition to be treated and the further selected therapeutic agents used in combined administration.

The terms “co-administration” or “combined administration” or the like as utilized herein are meant to encompass administration of the selected therapeutic agents to a single transplant recipient, and are intended to include treatment regimens in which the agents are not necessarily administered by the same route of administration or at the same time.

It is preferred that administration of the ALH agent, e.g., FTY720, be commenced preoperatively. In general, the compound may be administered starting from just prior to the day the transplant operation is carried out (i.e., “Day 0”), for example starting on Day −1, and continuing indefinitely thereafter. The compound may be administered, e.g., orally or by injection.

A preferred daily dosage range for the ALH agent, e.g., a compound of formula I (e.g. FTY720) is about from 0.03-2.5 mg/kg/day, particularly 0.1-2.5 mg/kg/day, e.g., 0.5-2.5 mg/kg/day as a single dose or in divided doses. Suitable daily dosages for patients are in the order of from, e.g., 0.25-100 mg p.o. Suitable unit dosage forms for oral administration of a compound of formula I comprise from ca. 0.125-10 mg together with one or more pharmaceutically acceptable diluents or carriers therefor. As an alternative, the compound of formula I in free form or in pharmaceutically acceptable salt form may also be administered twice or three times a week, e.g., at a dosage as indicated above. The ALH agent, e.g., the compounds of formula I, may be administered by any conventional route, in particular enterally, e.g., orally, for example, in the form of solutions for drinking, tablets or capsules or parenterally, for example, in the form of injectable solutions or suspensions. Pharmaceutical compositions comprising the compounds of formula I may be manufactured in conventional manner, e.g., as described in U.S. Pat. No. 5,604,229, incorporated herein by reference in its entirety.

The anti-IL2 receptor antibody, e.g., basiliximab, is preferably administered in a two-dose regimen, the first dose being administered on Day 0 (i.e., day of transplant) and a second on about Day 4. Additionally, doses following about Day 4 may optionally be administered, e.g., once weekly for 2 to 4 weeks. For primates, including humans, each dose is generally about 1-50 mg, and preferably about 5-20 mg.

The immunosuppressive macrocyclic lactone, e.g., 40-O-2-(hydroxyethyl)-rapamycin, is preferably administered on a daily basis, commencing on or just prior to the day of transplant (e.g., Day −1) and continuing on an indefinite basis following the transplant. For primates, including humans and non-human primates, suitable doses are in the range of 0.25-7 mg/day, and more particularly 0.5-5 mg/day. The compound may be administered orally or alternatively by subcutaneous (s.c.) injection.

The soluble complement receptor, e.g., TP-10, can be administered in single dosages of about 5-15 mg/kg, preferably about 10 mg/kg, as an i.v. infusion over about 30 minutes.

Most preferably, the invention is directed to a glucocorticoid-free combination therapy for use in connection with insulin-producing cell transplantation, e.g., pancreatic islet cell transplantation, comprising co-administration of an ALH agent, such as in particular a compound of formula I, e.g., 2-amino-2-[2-(4-octylphenyl)ethyl]propane-1,3-diol in free form or in a pharmaceutically acceptable salt form, preferably the hydrochloride salt thereof; in combination with one or more of basiliximab, 40-O-(2-hydroxyethyl)-rapamycin and the soluble recombinant human complement inhibitor, sCR1 (“TP10”).

The therapeutic methods of the invention may optionally include co-administration of still other immunomodulating drugs or anti-inflammatory agents, examples of which may comprise a calcineurin inhibitor, e.g., cyclosporins or ascomycins, and their immunosuppressive analogs, e.g., cyclosporin A, FK-506; cyclophosphamide; azathioprene; methotrexate; brequinar; leflunomide; mizoribine; mycophenolic acid; mycophenolate mofetil; 15-deoxyspergualine or analogues; immunosuppressive monoclonal antibodies, e.g., monoclonal antibodies to leukocyte receptors, e.g., to MHC, CD2, CD3, CD4, CD7, CD25, CD28, B7, CD40, CD45, or CD58 or to their ligands; or other immunomodulatory compounds, e.g., CTLA4-Ig or a homolog or mutant thereof, e.g., LEA29Y, or a LFA-1 inhibitor.

The methods of the invention may be employed as a prophylaxis or treatment of insulin-producing cell allograft or xenograft rejection.

The following example is to illustrate the invention, but should not be interpreted as a limitation thereon.

### EXAMPLE

### Transplantation of Allogeneic Islets into Cynomolgus Monkeys Suppressed with FTY720, Everolimus, Basiliximab and TP10

Therapeutic Agents:

FTY720: The compound is prepared for administration by emptying the contents of a capsule (1 mg/capsule) in a 60 mL clear glass mortar. 30 mL of sterile water are added and mixed with the capsule content until the powder is in a uniform suspension. The FTY720 is administered orally using a syringe and a nasogastric tube.

Basiliximab: The material is obtained as a package containing 20 mg of powder in a vial and a second vial containing 5 mL of diluent. Each vial is formulated according to the manufacturer's instructions and administered i.v. accordingly.

Everolimus: The compound is obtained as a concentrate of 20 mg/mL in a sealed ampoule. 1 mL of the concentrate is mixed with 8.5 mL vehicle (50% Cremophor and 50% ethanol) to give a final concentration of 2.1 mg/mL (pH 6.0) and the mixture is used within 2 hours.

TP10: The material is obtained as 50 mg multi-dose vials (no formulation required).
(a) Pancreatectomy of Donor Animals: Donor animals are adult cynomolgus monkeys over 4 kg, of either sex. Prior to pancreatectomy, fasting serum glucose analysis and aginine stimulation are performed to assure normal endocrine pancreatic function. For arginine stimulation, 0.07 mg/kg arginine are injected i.v., and blood collected at −5, −1, 2, 3, 4 and 5 minutes after arginine injection. Plasma is collected and stored frozen at −80° C. Plasma insulin and C-peptide levels are confirmed to be within normal range.
Pancreatectomy is performed under general anesthesia with 0.5-2% isofluorane, and the pancreas is harvested. Lymph nodes and spleen are also harvested for donor lymphocyte isolation and cryopreservation. Following organ and tissue harvest the donor is euthanized with 150 mg/Kg of Na pentobarbital i.v., and cardiac arrest confirmed by visual inspection.
(b) Islet Isolation: Islet isolation is performed using modifications of the automated method for human islet isolation, as described by Ricordi et al., Diabetes, Vol. 38, Suppl. 1, pp. 140-142 (1989); Kenyon et al., Diabetes, Vol. 48, pp. 8132-8137 (1999); and Ranuncoli et al., Cell Transplantation (2000).
Islet quality assessment is performed according to international standards (see Ricordi et al., Vol. 13 (1990)), including determination of islet yield, purity and viability. The number of islets obtained is reported as IEQ, which is the number of islets that would be present if the particles were all 150 μm in diameter. For this purpose, the number of dithizone (DTZ) positive islets in different size categories (50-100 μm, 100-150 μm, 150-200 μm, etc.) is counted and the data will be entered into a computer program that translates the information in IEQ. Purity is estimated based on the percentage of DTZ positive particles present in the preparation, and viability is estimated based on FDA/PI staining. In vitro functional capacity is determined via assessment of glucose stimulated insulin release in static cultures (see Ranuncoli et al., supra).
Transplant of Islet Allografts into Recipient Animals: Recipient animals are juvenile cynomolgus monkeys of >1.5 kg, of either sex. Recipient animals are rendered diabetic by infusion of streptozolocin (STZ), 150 mg/kg i.v., followed by i.v. hydration (20 mL/kg of 0.9% NaCl over 30 minutes) to prevent nephrotoxic side effects, as described by Thistlethwaite et al., Vol. 16 (1999). Blood glucose level is monitored frequently during the first 48 hours after STZ application to avoid severe hypoglycemia or hyperglycemia with eventual ketoacidosis. Thereafter, blood sugar levels are monitored 2-3 times daily and corrected with Regular, NPH, Lente, Ultralente, or Humalog insulin via s.c. injection or i.v. insulin-drip, as needed. Induction of diabetes is confirmed by daily blood glucose measurements, assessment of insulin requirements and by a negative C-peptide value subsequent to arginine stimulation. To confirm that diabetes has been induced, an arginine stimulation test is performed prior to initiation of immunosuppression. For arginine stimulation, 0.07 mg/kg arginine is injected intravenously, and blood collected −5, −1, 2, 3, 4 and 5 minutes after arginine injection. Plasma is collected and stored frozen at −80° C. Plasma glucose is measured using a Cobas Mira glucose analyzer (Roche Diagnostic Systems, Montclair, N.J.). A double antibody method (Diagnostic Products, Corp., Los Angeles, Calif.) is utilized to assess plasma insulin and C-peptide levels. The lower limit of detection for C-peptide is 0.20 ng/mL and for insulin is 5 uU/mL. Standard curves, as well as positive and negative control samples are incorporated into the assays.
Sedated and anesthetized recipient animals are placed supine on the operating table and the abdomen is prepped and draped. Special attention is paid to avoid hypothermia and a heat lamp and heating pads are used. A small central midline incision is made and a minimum of 10,000 freshly isolated islet equivalents/kg body weight are re-suspended in 20 mL of transplant media and infused into a mesenteric tributary of the portal vein through a 24-gauge i.v. catheter. To provide hemostasis, the vessel is either ligated or digital pressure is applied. The abdominal wall is closed in a routine fashion.
Blood glucose is monitored twice each day and arginine stimulation tests are performed to document reversal of the diabetic state.
Three groups of 4 recipients each are administered different immunosuppressive regimens and islet doses, as follows:
Group 1: FTY720+everolimus+basiliximab, 10,000 IEQ/kg body weight.
Group 2: FTY720+everolimus+basiliximab, 4,000 IEQ/kg body weight.
Group 3: FTY720+everolimus+basiliximab+TP10, 5,000 IEQ/kg body weight.
Recipient animals in the indicated groups receive the following treatments prior to and following the day of transplant (Day “0”) as detailed below:
FTY720 is administered p.o. at 0.3 mg/kg, day −1 through day +30.
Basiliximab is administered by i.v. injection at 10 mg on day 0 and day 4. Everolimus administered by s.c. injection once daily from day −1 through day +30, targeting trough levels of 15-30 ng/mL. The recommended dose is in the range of 0.075 mg/kg/day.
TP10 is administered by intravenous injection at a dosage level of 40 mg/kg on day 11, 20 mg/kg on days 0 and 1, and 17 mg/kg from days 2-7.
Drug monitoring is as follows:
FTY720—weekly. Everolimus—twice weekly. SC5b-9 is monitored for proof of TP10 efficacy.
The recipient animals are dosed at approximately the same time each morning and in the afternoon when applicable. Blood glucose levels are determined frequently over the first 4-5 hours post-transplant to prevent hypoglycemic episodes. Such episodes are treated with dextrose 5-10% i.v. as needed. The presence of rejection is suspected if three consecutive fasting blood glucose levels rise above 150 mg/dL or three post-prandial blood glucose levels of more than 200 mg/dL are recorded. Rejection is assumed if these levels are present 3 days in a row. Thereafter, fasting and post-prandial blood glucose (fasting blood glucose, post-prandial glucose) levels are monitored 2-3 times a day via heel stick. Daily blood glucose measurements plus periodic arginine stimulation tests (human insulin and C-peptide) at 14 and 30 days post-transplant are used to document glucose control. Exogenous insulin requirement after transplantation is also monitored. For the first 14 days after transplantation, blood glucose levels are corrected with Regular, NPH, Lente, or Ultralente Humalog insulin via s.c. injection according to an individualized sliding scheme. An arginine stimulation test is performed at 14 days and 1 month post-transplant and at additional time points thereafter.
(d) Pre-terminal Blood and Tissue Sampling: The transplant recipients are sedated by ketamine (5-10 mg/kg), intubated and pre-terminal blood sampling is performed under general anesthesia with 0.5-2% Isofluorane. The monkey is put in a supine position and the groin and abdomen are prepped and draped in a sterile fashion. The femoral vein, superior vena cava, or aorta is isolated, a catheter is placed, and 80-160 mL of blood are drawn. Samples of liver and spleen are harvested and frozen in liquid Nitrogen for RNA analysis. After completion of sterile sampling, the animal is euthanized by i.v. injection of sodium pentobarbital at a dose of 150 mg/kg. Necropsy samples are taken and fixed according to known procedures. Besides graft and organ samples, a sample of the pancreas is snap-frozen for insulin extraction.

The pathology of the graft organ is evaluated. Routine H&E stained sections are evaluated histologically. In this model, the combined treatment with FTY720, everolimus and basiliximab prevents islet allograft rejection.

1. A pharmaceutical combination comprising: a) 2-amino-2-[2-(4-octylphenyl)ethyl]propane-1,3-diol in free form or in pharmaceutically acceptable salt form; b) basiliximab, and c) 40-O-(2-hydroxyethyl)-rapamycin.

2. A combination according to claim 1 wherein a) is 2-amino-2-[2-(4-octylphenyl)ethyl]propane-1,3-diol in hydrochloride form.

3. A combination according to claim 1 further comprising a soluble human complement inhibitor.

4. A method for the treatment or prophylaxis of insulin-producing cell graft rejection in an insulin-producing cell graft recipient comprising co-administering to the recipient an effective amount of an ALH agent and one or more compounds selected from the group consisting of an antibody to the IL-2 receptor, an immunosuppressive macrocyclic lactone, and a soluble human complement inhibitor.

5. A method according to claim 4 wherein the ALH agent is a compound of formula I 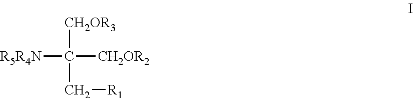
wherein
R₁ is an optionally substituted straight- or branched carbon chain having 12-22 carbon atoms which may be optionally interrupted by an optionally substituted phenylene;
R₂ is H or a residue of formula 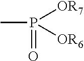
wherein
R₆ is H or C₁₋₄alkyl optionally substituted by 1, 2 or 3 halogen atoms, and R₇ is H or C₁₋₄alkyl optionally substituted by halogen;
R₃ is H or C₁₋₄alkyl; and
each of R₄ and R₅, independently, is H, C₁₋₄alkyl optionally substituted by halogen or acyl; in free form or in pharmaceutically acceptable salt form.

6. A method according to claim 4 wherein the ALH agent is 2-amino-2-[2-(4-octylphenyl)ethyl]propane-1,3-diol in free form or in a pharmaceutically salt form.

7. A method according to claim 4 wherein the antibody to the IL-2 receptor is a recombinant chimeric or humanized antibody.

8. A method according to claim 4 wherein the immunosuppressive macrocyclic lactone is rapamycin or a rapamycin derivative substituted in position 40 and/or 28 and/or 16, including epimers thereof, and optionally hydrogenated.

9. A method according to claim 4 wherein the immunosuppressive macrocyclic lactone is 40-O-(2-hydroxyethyl)-rapamycin.

10. A method according to claim 4 wherein the soluble human complement inhibitor is a C3/C5 inhibitor.
